(19) United States
(12) Patent Application Publication
(10) Pub. No.: US 2002/0028794 A1
(43) Pub. Date: Mar. 7, 2002

(54) Megestrol acetate suspension

(76) Inventors: Greg Allen Brubaker, Sunbury, OH (US); Mahendra Dedhiya, Nanuet, NY (US); Thomas Dean Mahon, West Jefferson, OH (US)

Correspondence Address: BOEHRINGER INGELHEIM CORPORATION, 900 RIDGEBURY ROAD, P O BOX 368, RIDGEFIELD, CT, 06877, US

(21) Appl. No.: 09/864,841

(22) Filed: May 24, 2001

Related U.S. Application Data
(60) Provisional application No. 60/219,822, filed on Jul. 21, 2000.

(51) Int. Cl. A61K 31/57

(52) U.S. Cl. 514/177

(57) ABSTRACT

An oral pharmaceutical composition in the form of a stable suspension in water, said composition comprising:

(a) about 10 to 200 mg per ml of micronized megestrol acetate;

(b) about 0.04 to 0.4% weight/volume of a wetting agent; and

(c) about 1.2 to 1.8% weight/volume of a suspending agent,

wherein said composition contains less than about 1.0% weight/volume of propylene glycol and does not contain polysorbate, polyethylene glycol, glycerol or sorbitol. The pH of the suspension can be controlled with an appropriate buffer system. In a preferred embodiment, the suspension has a pH of greater than about 3.3. The oral pharmaceutical composition is useful in the treatment of anorexia, cachexia or significant weight loss resulting from or associated with acquired immunodeficiency syndrome (AIDS).

### RELATED APPLICATIONS

Benefit of U.S. Provisional Application Serial No. 60/219,822, filed on Jul. 21, 2000, is hereby claimed.

### FIELD OF THE INVENTION

The present invention relates to a pharmaceutical composition containing megestrol acetate, which is the generic name for 17-α-acetyloxy-6-methylpregna-4,6 diene-3,20-dione. The pharmaceutical composition is in the form of a stable suspension in water, and is useful in the treatment of anorexia, cachexia or significant weight loss resulting from or associated with acquired immunodeficiency syndrome (AIDS).

### BACKGROUND OF THE INVENTION

Megestrol acetate is a synthetic derivative of the naturally occurring steroid hormone, progesterone. The megestrol acetate oral suspension Megace® (Bristol Myers Squibb Corp.) contains micronized megestrol acetate (40 mg/mL), polysorbate 80, polyethylene glycol, water and other conventional additives to form a flocculated suspension. Megace® oral suspension is currently approved for use in the treatment of anorexia, cachexia, or an unexplained, significant weight loss in patients with a diagnosis of acquired immunodeficiency syndrome (AIDS). It is desirable to have an oral liquid dosage form available to treat this patient group who may have difficulty swallowing tablets or capsules or where a high dose would require the ingestion of a relatively large number of tablets or capsules.

Megestrol acetate is insoluble in water therefore an oral suspension formulation is practical. According to Remington's Pharmaceutical Sciences (Chapter 21, page 313), a pharmaceutical suspension may be defined as a coarse dispersion containing finely divided insoluble material suspended in a liquid medium. Furthermore, a well formulated suspension should meet the following criteria. The dispersed particles should be of a size that they do not settle rapidly. If sedimentation occurs, however, the sediment must not form a hard cake but must be redispersable with a minimum of effort, i.e., a flocculated suspension. The product should also be easy to pour, pleasant to take, and resistant to microbial attack.

Possible concerns associated with the manufacture of suspensions are adequate dispersion of the particles in the vehicle, settling of the dispersed particles, and ultimate caking of the particles in the sediment making the suspension resistant to redispersion. In addition, the dissolution of the active drug substance and in vivo bioavailability is critical for a suspension dosage form and may be affected by the ingredients contained in the finished drug product. It is therefore desirable to define a formulation and process to address the issues described above to produce a pharmaceutically elegant oral suspension dosage form of megestrol acetate.

Kirk et al, U.S. Pat. No. 3,356,573, disclose a megestrol acetate pharmaceutical tablet preparation comprising lactose, magnesium stearate and starch. Kirk, et al, also disclose that liquid compositions of megestrol acetate can be useful but provided no details as to the composition of such formulations.

Petrow et al, U.S. Pat. No. 4,396,615, disclose a method of treating androgen-related disorders by administering 6-methyleneprogesterone derivatives concurrently with megestrol acetate. However, Petrow, et al. do not elaborate on what constitutes the megestrol acetate formulation.

Greaney et al., U.S. Pat. No. 4,370,321, disclose adjuvant therapy for the treatment of breast cancer employing megestrol acetate. However, the type or composition of the megestrol acetate formulation is not specifically described.

Labrie, U.S. Pat. No. 4,666,885, discloses combination therapy for treatment of female breast cancer comprising the administration of luteinizing hormones in combination with an anti-androgen compound such as megestrol acetate. In particular, Labrie discloses that the anti-androgens are formulated with conventional pharmaceutical excipients (e.g., spray dried lactose and magnesium stearate) into tablets or capsules for oral administration.

Labrie, U.S. Pat. No. 4,760,053 discloses methods of treating sex steroid dependent cancers by combination therapy which includes the use of megestrol acetate. However, Labrie does not describe the type or composition of pharmaceutical formulation used in the treatment.

Labrie, U.S. Pat. No. 4,775,661, discloses methods of treating female breast cancer with a combination therapy in which megestrol acetate is disclosed as a suitable steroidal anti-androgen. Labrie also discloses that megestrol acetate, as an active substance, may be mixed with lubricants such as polyethylene glycol and may include taste improving substances and can be worked into tablets or dragee cores.

Atzinger et al., U.S. Pat. No. 5,338,732, disclose an oral pharmaceutical composition of megestrol acetate in the form of a stable flocculated suspension of megestrol acetate in water. The flocculated suspension in Atzinger, et al, comprises megestrol acetate at a concentration of 15 to 150 mg/mL, in combination with a polysorbate at a concentration of 0.005% to 0.015% weight/volume (w/v) and a polyethylene glycol at a concentration greater than 5% w/v. Atzinger, et al., point out the distinction between a flocculated suspension and suspensions in general and point out that the stability of a flocculated suspension of a steroid such as megestrol acetate can be critically dependent on concentration.

Atzinger, et al., also disclose that megestrol acetate flocculated suspensions are unique because what would otherwise be predictable based on the prior art teachings does not apply when the drug is megestrol acetate. For instance, it is well known in the art prior to Atzinger et al. that megestrol acetate, a hydrophobic solid, is not easily wetted by water and has a relatively high interfacial tension accentuated by entrapped air absorbed on the surface of the particle. Hence, the use of surfactants are required to provide a suspension and maintain physical stability. According to Atzinger et al., the amount and type of surfactants are particularly critical in providing a stable floc. The flocculated suspension of megestrol acetate of Atzinger et al. uses megestrol acetate micronized so that 90% of the weight of particles is below 20 microns and the mass median diameter is between 3.0 and 10 microns, and requires that the micronized particles are dispersed in water with polysorbate and polyethylene glycol to reduce interfacial tension between the particle, entrapped gas and water.

Atzinger et al., discloses that in order to achieve a stable flocculated megestrol acetate suspension, polysorbate must be used at a concentration at about or less than 0.02% w/v, preferably from 0.005% to 0.015% w/v and most preferably 0.01% w/v, in combination with polyethylene glycol. At polysorbate 80 concentrations as low as 0.025% w/v, there is significant deflocculation and caking. They also state that at polysorbate concentrations at or below 0.005-0.01% w/v, a physically stable product was obtained but there is increased difficulty with respect to wetting of the micronized megestrol acetate at these low concentrations. Atzinger et al. further state that only surfactants having properties similar to polysorbate 80 can also be used. In this regard, they list polysorbate 20, 40, 60, 65 and 85 as having acceptable wetting properties.

Ragunathan, et al., U.S. Pat. No. 6,028,065, also disclose a stable flocculated suspension of megestrol acetate, but using a different formulation than that described in Atzinger. Specifically, Ragunathan, et al. disclose a stable flocculated suspension in water comprising:

a) megestrol acetate;

b) at least one compound selected from polyethylene glycol, propylene glycol, glycerol and sorbitol; and

c) a surfactant.

wherein polysorbate and polyethylene glycol are not simultaneously present in the composition. Ragunathan discloses that the compound selected from polyethylene glycol, propylene glycol, glycerol and sorbitol is preferably used at a concentration of up to 40% w/v, more preferably 5 to 30%, and most preferably 10 to 25%, and that the surfactant is preferably used at a concentration of about 0.0001 to 0.03% w/v. Ragunathan also discloses the use of a suspending agent (xanthan gum), preferably at a concentration of about 0.1 to 0.35% w/v, more preferably about 0.15 to 0.25%.

There exists a continuing need in the art for pharmaceutically elegant and stable oral suspension dosage forms of megestrol acetate.

### SUMMARY OF THE INVENTION

The present invention provides a pharmaceutically elegant and stable oral suspension dosage form of megestrol acetate that is different from known formulations and provides acceptable in-vitro dissolution performance.

In one embodiment, the present invention is directed to an oral pharmaceutical composition in the form of a stable suspension in water, said composition comprising:

(a) about 10 to 200 mg per ml of micronized megestrol acetate;

(b) about 0.04 to 0.4% weight/volume of a wetting agent; and

(c) about 1.2 to 1.8% weight/volume of a suspending agent,

wherein said composition contains less than about 1.0% weight/volume of propylene glycol and does not contain polysorbate, polyethylene glycol, glycerol or sorbitol. The pH of the suspension can be controlled with an appropriate buffer system. In a preferred embodiment, the suspension has a pH of at least about 3.3.

The oral pharmaceutical composition of the present invention is useful in the treatment of anorexia, cachexia or significant weight loss resulting from or associated with acquired immunodeficiency syndrome (AIDS).

### DETAILED DESCRIPTION OF THE INVENTION

The present invention is directed to an oral pharmaceutical composition in the form of a stable suspension in water, said composition comprising:

(a) about 10 to 200 mg per ml of micronized megestrol acetate;

(b) about 0.04 to 0.4% weight/volume of a wetting agent; and

(c) about 1.2 to 1.8% weight/volume of a suspending agent,

wherein said composition and does not contain polysorbate, polyethylene glycol, glycerol or sorbitol.

In one embodiment of the present invention, the concentration of megestrol acetate in the flocculated suspension is preferably about 10 to 200 mg per ml, more preferably about 20 to 60 mg per ml and most preferably about 40 mg per ml. It is preferred that the megestrol acetate is micronized so that 90% of the weight of particles is below 20 microns and the mass median diameter is between 3.0 and 10 microns.

The wetting agent (i.e., surfactant) of the present invention can be anionic or non-ionic.

Suitable anionic wetting agents include, for example, salts of sulphonic or monoesterified sulphuric acids such as alkyl benzene sulphonate, alkyl sulphates, alkyl ether sulphates, olefin sulphonates, alkyl phenol sulphates, alkyl phenol ether sulphates, alkyl ethanolamine sulphate, alkyl ethanolamine ether sulphates, alpha sulpho fatty acids or esters. Other suitable anionic wetting agents include fatty alkyl sulphosuccinates, fatty alkyl ether sulphosuccinates, acyl sarcosinates, acyl taurides, and paraffin sulphonates. The preferred anionic wetting agents are salts of alkali metals or alkaline earth metals, preferably docusate sodium, sodium lauryl sulfate and ducosate calcium. Other salts include ammonium, monoethanolamine, diethanolamine, triethanolamine and alkyl amines having up to 7 aliphatic carbon atoms.

Suitable non-ionic wetting agents include, for example, sorbitan fatty esters, polyoxyethylene stearates, polyoxyethylene alkyl ethers, polyethylene-propylene glycol copolymer (poloxamer), and polyoxyethylene castor oil derivatives. Preferred polyoxyethylene castor oil derivatives include polyoxyl 5 castor oil, polyoxyl 9 castor oil, polyoxyl 35 castor oil, polyoxyl 40 castor oil, polyoxyl 40 hydrogenated castor oil and polyoxyl 60 hydrogenated castor oil.

Wetting of a non-polar solute such as megestrol acetate takes place because it forms an association complex through Van der Waals interactions with a hydrophobic moiety of the surfactant (wetting agent). This effectively wets the material because the hydrophobic group of the surfactant is sequestering the megestrol while the hydrophilic group of the surfactant is solubilized in the bulk water.

In one embodiment of the present invention, the wetting agent of the present invention has a straight carbon chain of up to 20, preferably 4-20, atoms in the hydrophobic group. In a another embodiment, the straight carbon chain of the surfactant is less than 10 atoms, such as the one in ducosate.

The wetting agent of the present invention is present in the composition at a concentration of about 0.04 to 0.4% weight/volume, preferably at a concentration of about 0.04 to 0.1%. Conventional pharmaceutical carriers can be present, e.g., conventional suspending agents, protective colloids, preservatives, buffers, sweeteners/density adjusters and flavoring agents.

The use of a suspending agent maintains the megestrol acetate particles in a uniformly suspended state for a longer period of time during the dose administration period thereby permitting uniform dosing. Suitable suspending agents include, for example, xanthan gum, acacia gum, carrageenan, carbomer, sodium carboxymethylcellulose, hydroxyethyl cellulose, hydroxypropyl cellulose, hydroxypropylmethyl cellulose, and a mixture of microcrystalline cellulose and carboxymethylcellulose sodium. The suspending agent of the present invention is present in the composition at a concentration of about 1.2 to 1.8% weight/volume, preferably at a concentration of about 1.5 to 1.8%.

In some instances it may be desirable to use a protective colloid. The protective colloid is adsorbed onto the megestrol acetate particles and increases the strength of the hydration layer formed around suspended particles through hydrogen bonding and molecular interaction. This activity thus “protects” the megestrol acetate particles from binding to other particles and subsequent settling and caking. Suitable protective colloids include, for example, gelatin, acacia, carrageenan, carbomer, sodium carboxymethylcellulose, hydroxypropyl cellulose, hydroxypropylmethyl cellulose and xanthan gum. The protective colloid is generally present at a concentration of less than about 0.2% by weight/volume, preferably at a concentration of about 0.13 to 0.18%.

Suitable preservatives include, for example, sodium benzoate, benzoic acid, butylparaben, methylparaben, ethylparaben, propylparaben, phenylethyl alcohol, EDTA, benzyl alcohol, and phenoxyethanol. A preservative is typically used at a concentration of about 0.1 to 0.3% weight/volume, e.g., 0.15 to 0.25% weight/volume.

Suitable buffers (pH adjusters) include, for example, citric acid/sodium citrate, hydrochloric acid, succinic acid/sodium succinate, lactic acid, and potassium citrate. The buffer may be used to adjust the pH level of the suspension to a desired pH. In one embodiment, for example, a citric acid/sodium citrate is used where the citric acid level is held constant (e.g., 0.244%) and the pH is adjusted with sodium citrate (e.g., 0.015-0.11%).

In order to produce a non-flocculated (smooth) suspension, it is necessary that the pH of the suspension should be above a certain level, i.e., at least about 3.3. Another embodiment of the invention, therefore, is where the composition has a pH of at least about 3.3. By adjusting the pH in this manner, it is possible to obtain a suspension that is non-flocculated. It may be possible, however, that an initially non-flocculated suspension produced in this manner might develop some level of flocculation after being stored for a certain period of time. Of course, the present invention covers both the non-flocculated and flocculated forms, i.e., suspensions having any level of flocculation.

If the pH of the suspension is too high, however, there is a reduction in the effectiveness of sodium benzoate when used as a preservative in the formulation. Specifically, there is a reduction in the antimicrobial properties of sodium benzoate at high pH levels. Accordingly, if sodium benzoate is used as a preservative, it is preferable to keep the pH of the suspension at about 5.0 or less. Therefore, another embodiment of the present invention is where the composition has a pH of about 3.3 to 5.0, preferably about 3.6 to 4.6.

Suitable sweetening/density adjusting agents include, for example, sucrose, saccharin sodium, corn syrup and mannitol. A sweetening/density adjusting agent is typically used at a concentration of about 5 to 10% weight/volume, preferably about 5%. Flavoring agents are typically used at a concentration of about 0.35% weight/volume and include, for example, lemon and lemon-lime flavor.

The composition of the present invention contains less than about 1.0% weight/volume of propylene glycol, preferably less than about 0.5%, most preferably less than about 0.35%. If propylene glycol is present at all in the composition, it is generally present as an ingredient in the flavoring agent.

As apparent from the above discussion, various combinations of the wetting agents, suspending agents and other additives may be used in the formulation of the present invention, and certain specific ingredients may be added to perform special functions as needed. Although not specifically set forth herein, additional pharmaceutically acceptable carriers, adjuvants and additives may be employed, when necessary, to enhance stability or improve dissolution performance and bioavailability.

In a preferred embodiment, the wetting agent is polyoxyl 35 castor oil and the suspending agent is a mixture of microcrystalline cellulose and carboxymethylcellulose sodium.

A more specific embodiment of the oral pharmaceutical composition of the present invention is a stable suspension in water comprising about 40 mg of micronized megestrol acetate, about 0.04 to 0.4% weight/volume of polyoxyl 35 castor oil, about 1.2 to 1.8% weight/volume of a mixture of microcrystalline cellulose and carboxymethylcellulose sodium, about 0.15 to 0.25% weight/volume of sodium benzoate, about 0.24% weight/volume of citric acid, about 0.015 to 0.11% weight/volume of sodium citrate, about 5% weight/volume of sucrose, about 0.35% weight/volume of flavor, and the remainder water, wherein said composition contains less than about 0.35% weight/volume of propylene glycol and does not contain polysorbate, polyethylene glycol, glycerol or sorbitol. This more specific embodiment preferably has a pH of at least about 3.3, most preferably about 3.3 to 5.0.

The term “about” as used in the present specification and claims has the following meanings. When used in connection with a mg/mL value, the term “about” means within the range between 10% above and 10% below the noted value, inclusive (i.e., “about 40 mg/mL” means 36 to 44 mg/mL). When used in connection with a percent by weight/volume value, the term “about” means within the range between 10% above and 10% below the noted value, inclusive. When used in connection with a pH value, the term “about” means within the range between 0.1 pH units above and 0.1 pH units below the noted pH value, inclusive (i.e., “a pH of about 3.3” means a pH of 3.2 to 3.4). In addition, when the term “about” precedes a range of values, e.g., “about 10 to 200 mg per ml”, the term “about” modifies the indicated values at both ends of the range, e.g., “about 10 to 200 mg per ml” is equivalent to “about 10 to about 200 mg per ml”.

Exemplary formulations within the scope of the present invention are as set forth in the table below:

| Ingredient | Function | Each mL contains | |
|---|---|---|---|
| Megestrol acetate USP | Active ingredient | 40 | mg |
| (micronized) | | | |
| Polyoxyl 35 castor oil, NF | Wetting agent | 0.04-0.4% | w/v |
| Microcrystalline cellulose and | Suspending agent | 1.2-1.8% | w/v |
| carboxymethylcellulose sodium, | | | |
| NF | | | |
| Sodium benzoate, NF (powder) | Preservative | 0.15-0.25% | w/v |

| | | |
|---|---|---|
| Citric acid, USP | Buffer | amount adjusted to |
| (anhydrous, fine granular) | | target pH |
| Sodium citrate, USP (granular) | Buffer | amount adjusted to |
| | | target pH |
| Sugar, fine granular | Sweetening agent/ | q.s. to taste/ |
| (sucrose, NF) | Density Adjuster | density |
| Flavor | Flavorant | q.s. to taste |
| Water, purified, USP | Solvent/Vehicle | q.s. |

The pharmaceutical composition of the present invention as a stable suspension in water can be prepared using conventional techniques well known to those having ordinary skill in the art.

An exemplary manufacturing process is set forth below:

1) Add the suspending agent to a portion of water, purified, USP and mix well.

2) Add the wetting agent and active ingredient to another portion of water, purified, USP and mix well.

3) Add the preservative to another portion of water, purified, USP and mix well.

4) Add the sweetening agent and buffers to another portion of water, purified, USP and mix well.

5) Add Step 3 to Step 1 and mix well.

6) Add Step 4 to Step 5 and mix well.

7) Add the flavorant to Step 6 and mix well.

8) Add Step 2 to Step 7 and mix well.

9) Bring Step 8 to volume with water, purified, USP and mix well.

10) Pass Step 9 through a mill or homogenizer into a storage tank.

Further exemplary manufacturing processes are set forth in the specific examples below.

The oral pharmaceutical composition of the present invention is useful in the treatment of anorexia, cachexia or significant weight loss resulting from or associated with acquired immunodeficiency syndrome (AIDS).

The appropriate dosage levels and requirements for megestrol acetate in these indications are well-recognized in the art and may be selected by those of ordinary skill in the art from available methods and techniques suitable for a particular patient. A suitable adult oral dosage of megestrol acetate might be in the range from about 100 to 1000 mg/day, depending on the particular patient. The required daily dosage of megestrol acetate may be administered in single or multiple doses. It should be understood, however, that the dosage administration from patient to patient will vary and the dosage for any particular patient will depend upon the clinician's judgment, who will use as criteria for fixing a proper dosage the size and condition of the patient as well as the patient's response to the treatment regimen.

In-vitro dissolution studies of megestrol acetate have shown that it is properly solubilized with the pharmaceutical composition of the present invention. Physical stability on a representative sample of the composition of the present invention was studied at 40° C. and 75% relative humidity. The physical stability of the product after storage for 3 months was acceptable.

The present invention is illustrated in further detail by the following non-limiting examples:

### EXAMPLE 1

A lemon-lime flavored oral suspension containing 40 mg of megestrol acetate per milliliter was prepared with a list of ingredients according to the below table.

### Formulation for Megestrol Acetate Suspension

| Ingredient | Each mL contains | |
|---|---|---|
| Megestrol acetate USP | 40 | mg |
| (micronized) | | |
| Polyoxyl 35 Castor Oil, NF | 0.04% | w/v |
| Microcrystalline cellulose and | 1.8% | w/v |
| carboxymethylcellulose | | |
| sodium, NF | | |
| Sodium benzoate, NF (powder) | 0.20% | w/v |
| Citric acid, USP | 0.244% | w/v |
| (anhydrous, fine granular) | | |
| Sodium citrate, USP (granular) | 0.11% | w/v |
| Sugar, fine granular (sucrose, | 5.0% | w/v |
| NF) | | |
| Lemon-Lime Flavor | 0.35% | w/v |

| | |
|---|---|
| Water, purified, USP | q.s. to volume |

Preparation of the megestrol acetate suspension using the above proportional amounts of ingredients is carried out as follows. The microcrystalline cellulose/carboxymethylcellulose sodium (MCC/CMC) powder is uniformly hydrated in a portion of water using a colloid mill or homogenizer. The sodium benzoate is dissolved in a portion of water and added to the MCC/CMC dispersion. The citric acid and sodium citrate are dissolved in another portion of water. The sugar is then added to the citric acid/sodium succinate solution and dissolved. The resultant citric acid, sodium citrate, and sugar solution is added to the MCC/CMC dispersion. The lemon-lime flavor is added to the MCC/CMC dispersion. The polyoxyl 35 castor oil is added to a portion of water to form a solution. The megestrol acetate is then dispersed in the polyoxyl 35 castor oil solution. The megestrol dispersion is added to the MCC/CMC dispersion. The entire suspension is passed through a colloid mill or homogenizer until a uniform suspension is obtained.

### EXAMPLE 2

A lemon-lime flavored oral suspension containing 40 mg of megestrol acetate per milliliter was prepared with a list of ingredients according to the below table.

### Formulation for Megestrol Acetate Suspension

| Ingredient | Each mL contains | |
|---|---|---|
| Megestrol acetate USP | 40 | mg |
| (micronized) | | |
| Polyoxyl 35 Castor Oil, NF | 0.1% | w/v |
| Microcrystalline cellulose and | 1.3% | w/v |
| carboxymethylcellulose | | |
| sodium, NF | | |
| Sodium benzoate, NF (powder) | 0.20% | w/v |
| Citric acid, USP | 0.244% | w/v |
| (anhydrous, fine granular) | | |
| Sodium citrate, USP (granular) | 0.015% | w/v |
| Sugar, fine granular (sucrose, | 5.0% | w/v |
| NF) | | |
| Lemon-Lime Flavor | 0.35% | w/v |

| | |
|---|---|
| Water, purified, USP | q.s. to volume |

Preparation of the megestrol acetate suspension using the above proportional amounts of ingredients is carried out as follows. The microcrystalline cellulose/carboxymethylcellulose sodium (MCC/CMC) powder is uniformly hydrated in a portion of water using a colloid mill or homogenizer. The sodium benzoate is dissolved in a portion of water and added to the MCC/CMC dispersion. The citric acid, sodium citrate, and sugar are dissolved in another portion of water. This solution is added to the MCC/CMC dispersion. The lemon-lime flavor is added to the MCC/CMC dispersion. The polyoxyl 35 castor oil is added to a portion of water to form a solution. The megestrol acetate is then dispersed in the polyoxyl 35 castor oil solution. The megestrol dispersion is added to the MCC/CMC dispersion. The entire suspension is passed through a colloid mill or homogenizer until a uniform suspension is obtained.

### EXAMPLE 3

An oral suspension containing 40 mg of megestrol acetate per milliliter was prepared with a list of ingredients according to the below table.

### Formulation for Megestrol Acetate Suspension

| Ingredient | Each mL contains | |
|---|---|---|
| Megestrol acetate USP | 40 | mg |
| (micronized) | | |
| Polyoxyl 35 Castor Oil, NF | 0.4% | w/v |
| Microcrystalline cellulose and | 1.8% | w/v |
| carboxymethylcellulose | | |
| sodium, NF | | |
| Xanthan gum, NF | 0.18% | w/v |
| Sodium benzoate, NF (powder) | 0.20% | w/v |
| Citric acid, USP | 0.244% | w/v |
| (anhydrous, fine granular) | | |
| Sodium citrate, USP (granular) | 0.015% | w/v |
| Sugar, fine granular (sucrose, | 5.0% | w/v |
| NF) | | |

| | |
|---|---|
| Water, purified, USP | q.s. to volume |

Preparation of the megestrol acetate suspension using the above proportional amounts of ingredients is carried out as follows. The microcrystalline cellulose/carboxymethylcellulose sodium (MCC/CMC) and xanthan gum powders are uniformly hydrated in a portion of water using a colloid mill or homogenizer. The citric acid, sodium citrate, and sugar are dissolved in another portion of water. This solution is added to the MCC/CMC dispersion. The sodium benzoate is dissolved in a portion of water and added to the MCC/CMC dispersion. The polyoxyl 35 castor oil is added to a portion of water to form a solution. The megestrol acetate is then dispersed in the polyoxyl 35 castor oil solution. The megestrol dispersion is added to the MCC/CMC dispersion. The pH of the suspension is adjusted with hydrochloric acid as necessary. The product is brought to volume with water. The entire suspension is passed through a colloid mill or homogenizer until a uniform suspension is obtained.

What is claimed is:

1. An oral pharmaceutical composition in the form of a stable suspension in water, said composition comprising:
(a) about 10 to 200 mg per ml of micronized megestrol acetate;
(b) about 0.04 to 0.4% weight/volume of a wetting agent; and
(c) about 1.2 to 1.8% weight/volume of a suspending agent,
wherein said composition contains less than about 1.0% weight/volume of propylene glycol and does not contain polysorbate, polyethylene glycol, glycerol or sorbitol.

2. A composition according to claim 1, wherein the concentration of micronized megestrol acetate is about 40 mg per ml.

3. A composition according to claim 1, wherein the wetting agent comprises a hydrophobic moiety having a straight carbon chain of up to 20 atoms.

4. A composition according to claim 1, wherein the wetting agent is non-ionic.

5. A composition according to claim 4, wherein the wetting agent is selected from the group consisting sorbitan fatty esters, polyoxyethylene stearates, polyoxyethylene alkyl ethers, polyethylene-propylene glycol copolymer, and polyoxyethylene castor oil derivatives.

6. A composition according to claim 5, wherein the wetting agent is selected from the group consisting of polyoxyl 5 castor oil, polyoxyl 9 castor oil, polyoxyl 35 castor oil, polyoxyl 40 castor oil, polyoxyl 40 hydrogenated castor oil and polyoxyl 60 hydrogenated castor oil.

7. A composition according to claim 1, wherein the wetting agent is anionic.

8. A composition according to claim 7, wherein the wetting agent is selected from the group consisting of salts of sulphonic acids, fatty alkyl sulphosuccinates, fatty alkyl ether sulphosuccinates, acyl sarcosinates, acyl taurides, paraffin sulphonates, salts of alkali metals and salts of alkaline earth metals.

9. A composition according to claim 7, wherein the wetting agent is selected from the group consisting of sodium lauryl sulfate, docusate sodium and docusate calcium.

10. A composition according to claim 1, wherein the suspending agent is selected from the group consisting of xanthan gum, acacia gum, carrageenan, carbomer, sodium carboxymethylcellulose, hydroxyethyl cellulose, hydroxypropyl cellulose, hydroxypropylmethyl cellulose, and a mixture of microcrystalline cellulose and carboxymethylcellulose sodium.

11. A composition according to claim 1, wherein said composition has a pH of at least about 3.3.

12. A composition according to claim 11, wherein said composition has a pH of about 3.3 to 5.0.

13. A composition according to claim 1, further comprising a protective colloid in an amount of less than about 0.2% by weight/volume.

14. A composition according to claim 13, wherein the protective colloid is selected from the group consisting of gelatin, acacia, carrageenan, carbomer, sodium carboxymethylcellulose, hydroxypropyl cellulose, hydroxypropylmethyl cellulose and xanthan gum.

15. A composition according to claim 1, wherein the wetting agent is polyoxyl 35 castor oil and wherein the suspending agent is a mixture of microcrystalline cellulose and carboxymethylcellulose sodium.

16. An oral pharmaceutical composition in the form of a stable suspension in water comprising about 40 mg of micronized megestrol acetate, about 0.04 to 0.4% weight/volume of polyoxyl 35 castor oil, about 1.2 to 1.8% weight/volume of a mixture of microcrystalline cellulose and carboxymethylcellulose sodium, about 0.15 to 0.25% weight/volume of sodium benzoate, about 0.24% weight/volume of citric acid, about 0.015 to 0.11% weight/volume of sodium citrate, about 5% weight/volume of sucrose, about 0.35% weight/volume of flavor, and the remainder water, wherein said composition contains less than about 0.35% weight/volume of propylene glycol and does not contain polysorbate, polyethylene glycol, glycerol or sorbitol.

17. A composition according to claim 16, wherein said composition has a pH of at least about 3.3.

18. A composition according to claim 17, wherein said composition has a pH of about 3.3 to 5.0.

19. A method for the treatment of anorexia, cachexia or significant weight loss resulting from or associated with acquired immunodeficiency syndrome, which comprises administering to a host in need of such treatment a therapeutic amount of the oral pharmaceutical composition according to claim 1.

20. A method for the treatment of anorexia, cachexia or significant weight loss resulting from or associated with acquired immunodeficiency syndrome, which comprises administering to a host in need of such treatment a therapeutic amount of the oral pharmaceutical composition according to claim 16.
